(19) United States
(12) Patent Application Publication
(10) Pub. No.: US 2023/0065595 A1
(43) Pub. Date: Mar. 2, 2023

(54) DETECTION OF POLYMER SHALE ENCAPSULATORS IN WELLBORE FLUID

(71) Applicant: Halliburton Energy Services, Inc., Houston, TX (US)

(72) Inventors: Preston May, Houston, TX (US); Jay Deville, Houston, TX (US)

(21) Appl. No.: 17/459,257

(22) Filed: Aug. 27, 2021

(51) Int. Cl. G01N 21/82; G01N 21/47; G01N 33/28

(52) U.S. Cl. CPC G01N 21/82; G01N 21/47; G01N 33/2823

(57) ABSTRACT

Systems and methods to determine concentrations of certain polymer encapsulators in drilling fluids based on light scattering, turbidity analysis and other analysis techniques. The method generally includes disposing a sample of drilling fluid within a vessel. A precipitant is added to form a precipitate with the desired polymer resulting in turbidity which is then measured in a variety of ways to determine the amount of polymer in the drilling fluid.

### FIELD OF THE INVENTION

The present invention relates generally to testing systems for wellbore fluids and, more specifically, to a method and testing system to detect polymers in wellbore water-based mud.

### BACKGROUND

Polymers are frequently used as shale encapsulators in water-based drilling fluids. These polymer products can coat the surface of clay particles creating a barrier that slows the diffusion of water into the clay pore spaces. Encapsulators generally mitigate clay dispersion and have a positive impact on cuttings stability leading to better hole cleaning and less buildup of clay fines in the fluid over time. These products are used in a proactive manner and their typical use leads depletion over time. If no action is taken, eventually the concentration of these polymers will go to zero.

Conventional drilling methods experience decreasing levels of encapsulator concentrations over time. Further, these methods do not monitor the active concentration of these compounds while drilling. Therefore, this uncertainty can lead to diminished service quality and lost time and money.

### BRIEF DESCRIPTION OF THE DRAWINGS

FIG. 1 illustrates a drilling application in which the illustrative methods of the present disclosure may be applied.

FIG. 2 is a flow chart of a method to determine an amount of polymers in a drilling fluid, according to one or more illustrative methods of the present disclosure.

FIG. 3 illustrates five sample vessels having fluid therein with varying amounts of turbidity.

FIG. 4 illustrates one example of a turbidity meter which can be used in illustrative embodiments of the present disclosure.

FIG. 5 is a graph showing a calibration curve between turbidity measurements and PVP, according to certain illustrative methods of the present disclosure.

FIG. 6 is a table and graph illustrating how no interference is observed (with amine shale inhibitors) on the disclosed precipitation analysis methods.

FIG. 7 is a block diagram of an exemplary computer system in which embodiments of the present disclosure may be implemented.

### DESCRIPTION OF ILLUSTRATIVE EMBODIMENTS

Illustrative embodiments and related methods of the present disclosure are described below as they might be employed to determine concentrations of polymer encapsulators in wellbore fluids using light scattering, turbidity analysis and/or other methods. In the interest of clarity, not all features of an actual implementation or methodology are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. Further aspects and advantages of the various embodiments and related methodologies of the invention will become apparent from consideration of the following description and drawings.

Exemplary embodiments of the present invention are directed to systems and methods to determine concentrations of certain polymer encapsulators in drilling fluids based using light scattering and turbidity analysis methods. The polymers may be, for example, a polymer that is a linear, crosslinked or branched polyvinlypyrrolindone (“PVP”) homopolymer or a linear, crosslinked or branched co-polymer containing at least a vinylpyrrolidone monomer (i.e., polymers that contain the vinylpyrrolidone monomer). In a generalized method, a sample of a drilling fluid having an aqueous base fluid and PVP, for example, is obtained within a vessel. A precipitant is added to the sample to form a precipitate with the PVP. The precipitant may be, for example, resorcinol, resorcylic acid, or tannic acid. The precipitate is separated from the aqueous base fluid, whereby the amount of PVP within the sampled fluid is determined through measurement of the precipitate within the sample. The precipitate may be measured using a variety of techniques including, for example, by performing a turbidity analysis of the sample using scattered light, by gravimetric weight analysis of the precipitate, or by volumetric analysis of the precipitate.

The test solution, a fluid suspension containing the precipitate, can be analyzed to determine the amount of precipitate formed. In some examples, the test solution is analyzed using a turbidity meter to measure the amount of precipitate in the test solution and determine the amount of PVP that was present in the sample of drilling or other fluid. In other examples, the test solution can be filtered to remove and dry the precipitate, which is then weighed to measure the amount of precipitate in the test solution and determine the amount of PVP that was present in the sample of drilling fluid. In other examples, the solids can be allowed to settle in a graduated vessel and the volume of precipitate formed measured to determine the amount of PVP that was present in the sample of drilling or other fluid.

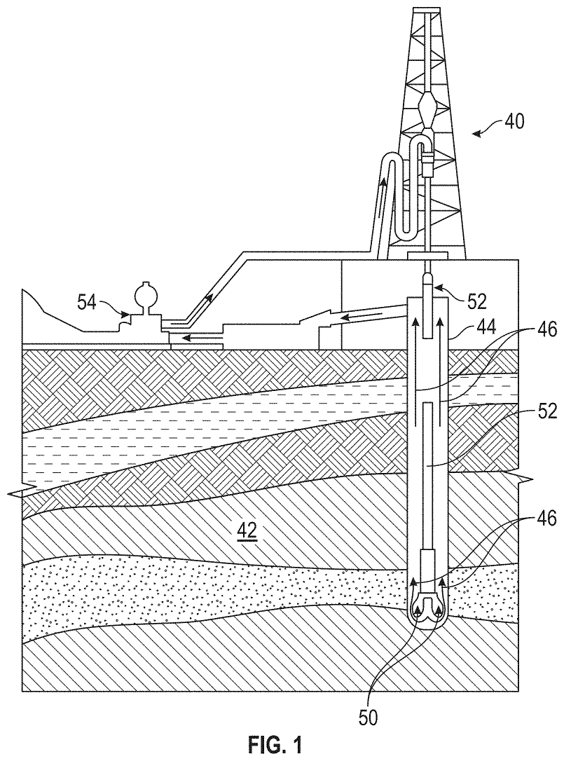

FIG. 1 illustrates a drilling application in which the illustrative methods of the present disclosure may be applied. Wellbore 44 is being drilled through a subterranean formation 42. A drill rig 40 can be used for drilling the wellbore 44. A drill bit 50 may be mounted on the end of a drill string 52 that includes multiple sections of drill pipe. The wellbore 44 may be drilled by using a rotary drive at the surface to rotate the drill string 52 and to apply torque and force to cause the drill bit 50 to extend through wellbore 44. A drilling fluid may be displaced through the drill string 52 using one or more pumps 54. The drilling fluid may be circulated past the drill bit 50 and returned to the surface through the annulus of wellbore 44, as indicated by arrows 46, thereby removing drill cuttings (e.g., material such as rock generated by the drilling) from the wellbore 44. A shale encapsulator, such as PVP, can be added to the drilling fluid. Although not shown, additional conduits besides drill string 52 may also be disposed within wellbore 44.

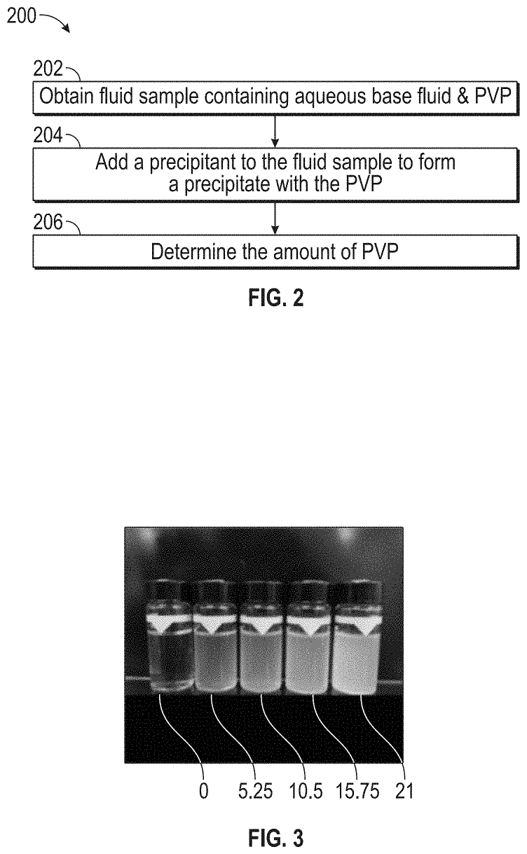

FIG. 2 is a flow chart of a method to determine an amount of PVP in a drilling fluid, according to one or more illustrative methods of the present disclosure. Note this example is directed to PVP in particular. However, this illustrative method may also be applied to other polymers, such as those described herein. In block 202, a sample of a drilling fluid is obtained (in a vessel or other sampling container) which contains an aqueous base fluid and a polymer (e.g., a linear, crosslinked, or branched polyvinlypyrrolindone (“PVP”) or a linear, crosslinked or branched co-polymer containing at least a vinylpyrrolidone monomer (or a derivative thereof)). The solids are removed from the sample to produce a solids-free fluid. The solids can be removed from the drilling fluid sample using any suitable device and/or method. Examples of suitable solids removal devices and methods include filtration, centrifugation, simple settling, dissolution or chemical extraction. Testing may be conducted on at least a portion of the solids-free fluid, as described below.

Polyphenolic compounds react with the polymer (e.g., PVP) to create insoluble particles that generate turbid suspensions. The turbidity can be used to determine the concentration of the polymer in the fluid. FIG. 3 illustrates five sample vessels having fluid therein with varying amounts of turbidity. At block 204, a precipitant is added to the sample to form a precipitate with at least a portion of the polymer (FIG. 3 shows separation of precipitate from the fluid resulting in the turbidity of the fluid sample), resulting in the turbidity. The precipitant may take a variety of forms such as, for example, zinc or a polyphenolic compound such as resorcinol, resorcylic acid, or tannic acid. At block 206, the system then determines the amount of polymer within the water-based mud by measuring an amount of precipitate within the sample (e.g., via turbidity analysis) using an analyzing device. In alternate embodiments, the precipitate may be separated from the aqueous fluid and gravimetrically or volumetrically weighed (or otherwise analyzed) to determine the amount of precipitate in the sample.

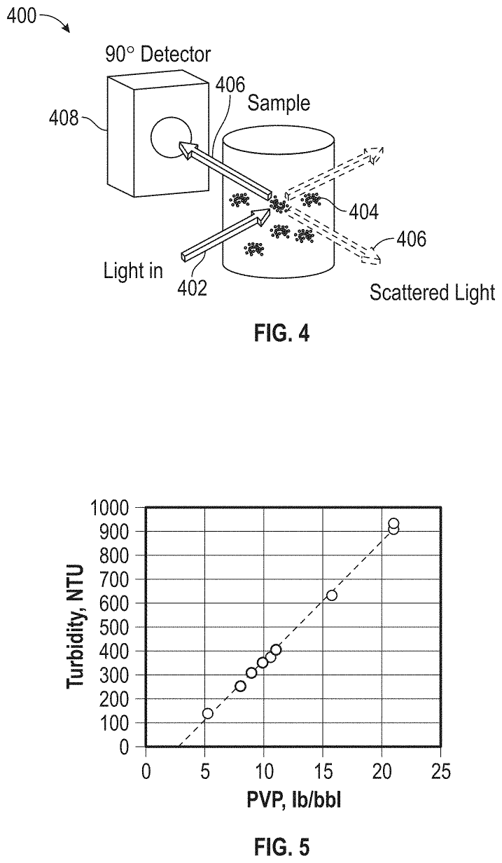

In block 206, the precipitate can be measured in a variety of ways. In certain illustrative embodiments, the precipitate is measured using an analyzer such as a turbidity meter, gravimetric weight analyzer, spectrophotometer (which measures absorbance or transmittance of light through the turbid sample) or volumetric analyzer. FIG. 4 illustrates one example of a turbidity meter 400 which supplies electromagnetic radiation 402 to the precipitate 404.

Scattered light 406 is then generated and measured by detector 408. A variety of turbidity meters may be used, all of which will output a turbidity measurement (measured in Nephelometric Turbidity Units). FIG. 5 is a graph showing a calibration curve between turbidity measurements and PVP.

Using a graph such as in FIG. 5, the measured amount of precipitate (e.g., the PVP-precipitant complex) can then be correlated to known values and concentrations of PVP. Turbidity measured in NTUs is correlated to product the concentration in lb/bbl (pounds of product per barrel of fluid). The higher the concentration of product in the sample, the higher the turbidity in NTUs. A calibration curve is used to convert a specific NTU value to a specific product concentration value. Once a calibration curve is established as in FIG. 5, any turbidity value measured (Y-axis) with a drilling fluid can be converted to a concentration of PVP (X-axis).

Further description of the calibration curve technique will now be provided. In one example, a calibration curve is made with drilling fluids with known concentrations of PVP or other desired polymer (lb/bbl). The test is performed on each drilling fluid to obtain a turbidity value (NTU) for that concentration of PVP. Turbidity values (NTU) as a function of PVP concentration (lb/bbl in drilling fluid) are then plotted to obtain a calibration curve establishing the relationship between turbidity (NTU) and polymer concentration (lb/bbl in drilling fluid). Thereafter, any drilling fluid can be tested and the turbidity value that results from performing the test can be converted directly to polymer concentration in drilling fluid using the calibration curve. The concentration of polymer in the fluid sample is also the concentration of polymer in the drilling fluid overall.

With regard to the gravimetric method, the precipitate can be removed from the fluid, dried and weighed. The higher the mass measured in, for example, mg or g, the higher the concentration of the polymer in the drilling fluid. With regard to volumetric measurements, the precipitate would be allowed to settle in a graduated vessel. The higher volume of a precipitate would lead to a higher measured height on a graduated vessel. The higher the volume of precipitate, the higher the concentration of polymer in a drilling fluid. Lastly, with regard to an illustrative spectroscopic method, the suspension containing the precipitate could be subjected to an incident light beam with a wavelength anywhere in the ultraviolet or visible spectrum. The precipitate particles would absorb light (or alternatively, reduce transmission of light) leading to a lower absorption of light compared to a sample with no precipitate. The higher the absorption of light, the higher the concentration of polymer in the fluid.

Thereafter, the drilling or other wellbore operation may be modified or otherwise adjusted based upon the measured amount of polymer. In some examples, this may require adding more polymer to the drilling mud in real-time or changing other properties or ingredients of the drilling mud. Such a system may be completed automated to make the added polymer injection to the drilling mud. In other examples, the polymer may be added to the drilling mud manually.

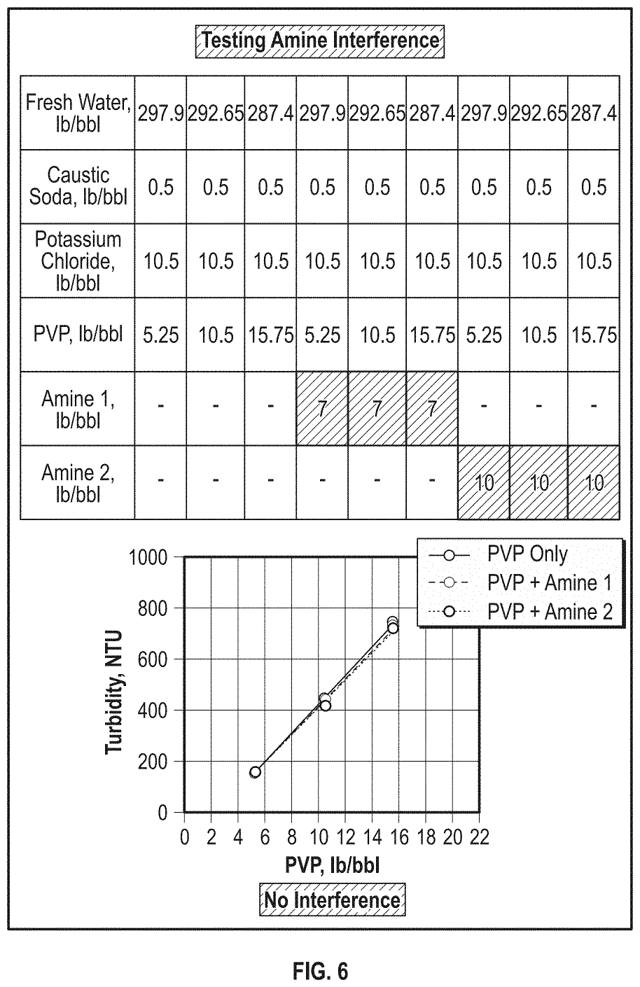

FIG. 6 is a table and graph illustrating how no interference is observed (with amine shale inhibitors) on the disclosed precipitation analysis methods to detect PVP (in this example) in aqueous solutions. As can be seen, the test results, in terms of turbidity, are the same for all samples of equal PVP concentration even in the presence of other amine-containing products. In other words, the presence of other amine-containing products does not change the test results (i.e. no interference).

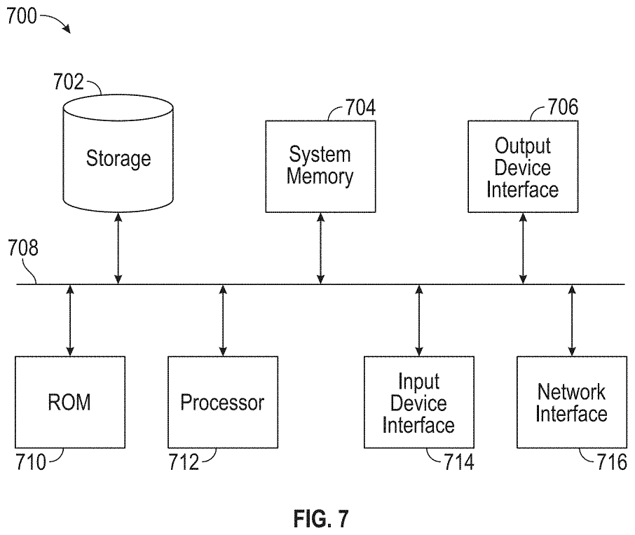

FIG. 7 is a block diagram of an exemplary computer system 700 in which embodiments of the present disclosure may be implemented. System 700 can be a computer, phone, PDA, or any other type of electronic device. Such an electronic device includes various types of computer readable media and interfaces for various other types of computer readable media. As shown in FIG. 7, system 700 includes a permanent storage device 702, a system memory 704, an output device interface 706, a system communications bus 708, a read-only memory (ROM) 710, processing unit(s) 712, an input device interface 714, and a network interface 716.

Bus 708 collectively represents all system, peripheral, and chipset buses that communicatively connect the numerous internal devices of system 700. For instance, bus 708 communicatively connects processing unit(s) 712 with ROM 710, system memory 704, and permanent storage device 702.

From these various memory units, processing unit(s) 712 retrieves instructions to execute and data to process in order to execute the processes of the subject disclosure. The processing unit(s) can be a single processor or a multi-core processor in different implementations.

ROM 710 stores static data and instructions that are needed by processing unit(s) 712 and other modules of system 700. Permanent storage device 702, on the other hand, is a read-and-write memory device. This device is a non-volatile memory unit that stores instructions and data even when system 700 is off. Some implementations of the subject disclosure use a mass-storage device (such as a magnetic or optical disk and its corresponding disk drive) as permanent storage device 702.

Other implementations use a removable storage device (such as a floppy disk, flash drive, and its corresponding disk drive) as permanent storage device 702. Like permanent storage device 702, system memory 704 is a read-and-write memory device. However, unlike storage device 702, system memory 704 is a volatile read-and-write memory, such a random access memory. System memory 704 stores some of the instructions and data that the processor needs at runtime. In some implementations, the processes of the subject disclosure are stored in system memory 704, permanent storage device 702, and/or ROM 710. For example, the various memory units include instructions for computer aided pipe string design based on existing string designs in accordance with some implementations. From these various memory units, processing unit(s) 712 retrieves instructions to execute and data to process in order to execute the processes of some implementations.

Bus 708 also connects to input and output device interfaces 714 and 706. Input device interface 714 enables the user to communicate information and select commands to the system 700. Input devices used with input device interface 814 include, for example, alphanumeric, QWERTY, or T9 keyboards, microphones, and pointing devices (also called “cursor control devices”). Output device interfaces 706 enables, for example, the display of images generated by the system 700. Output devices used with output device interface 706 include, for example, printers and display devices, such as cathode ray tubes (CRT) or liquid crystal displays (LCD). Some implementations include devices such as a touchscreen that functions as both input and output devices. It should be appreciated that embodiments of the present disclosure may be implemented using a computer including any of various types of input and output devices for enabling interaction with a user. Such interaction may include feedback to or from the user in different forms of sensory feedback including, but not limited to, visual feedback, auditory feedback, or tactile feedback. Further, input from the user can be received in any form including, but not limited to, acoustic, speech, or tactile input. Additionally, interaction with the user may include transmitting and receiving different types of information, e.g., in the form of documents, to and from the user via the above-described interfaces.

Also, as shown in FIG. 7, bus 708 also couples system 700 to a public or private network (not shown) or combination of networks through a network interface 716. Such a network may include, for example, a local area network (“LAN”), such as an Intranet, or a wide area network (“WAN”), such as the Internet. Any or all components of system 700 can be used in conjunction with the subject disclosure.

These functions described above can be implemented in digital electronic circuitry, in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself. Accordingly, the steps of processes described above may be implemented using system 700 or any computer system having processing circuitry or a computer program product including instructions stored therein, which, when executed by at least one processor, causes the processor to perform functions relating to these methods.

As used in this specification and any claims of this application, the terms “computer”, “server”, “processor”, and “memory” all refer to electronic or other technological devices. These terms exclude people or groups of people. As used herein, the terms “computer readable medium” and “computer readable media” refer generally to tangible, physical, and non-transitory electronic storage mediums that store information in a form that is readable by a computer.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., a web page) to a client device (e.g., for purposes of displaying data to and receiving user input from a user interacting with the client device). Data generated at the client device (e.g., a result of the user interaction) can be received from the client device at the server.

It is understood that any specific order or hierarchy of steps in the processes disclosed is an illustration of exemplary approaches. Based upon design preferences, it is understood that the specific order or hierarchy of steps in the processes may be rearranged, or that all illustrated steps be performed. Some of the steps may be performed simultaneously. For example, in certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Embodiments and methods of the present disclosure described herein further relate to any one or more of the following paragraphs:

1. A method to determine a polymer concentration in an aqueous drilling fluid, the method comprising obtaining a sample of a drilling fluid comprising an aqueous base fluid and a polymer comprising a vinylpyrrolidone monomer; adding a precipitant to the sample to form a precipitate comprising at least a portion of the polymer; and determining an amount of the polymer within the drilling fluid by performing at least of a turbidity analysis of the sample, a gravimetric weight analysis of the precipitate, a volumetric analysis of the precipitate or combinations thereof.

2. The method of paragraph 1, wherein the precipitant is selected from polyphenolic compounds, zinc compounds or combinations thereof.

3. The method of paragraphs 1 or 2, wherein determining the amount of polymer comprises separating the precipitate from the aqueous base fluid by removing solids from the drilling fluid; and measuring a turbidity of the sample.

4. The method of any of paragraphs 1-3, further comprising, in response to the determined concentration of the polymer, adding additional polymer to the drilling fluid.

5. The method of any of paragraphs 1-4, wherein the polymer is a linear, crosslinked, or branched polyvinlypyrrolindone (“PVP”) homopolymer or a linear, crosslinked or branched co-polymer containing at least a vinylpyrrolidone monomer.

6. A system to determine a concentration of vinlypyrrolindone in drilling fluid, the system comprising a vessel to obtain a sample of drilling fluid comprising an aqueous base fluid and a polymer comprising a vinylpyrrolidone monomer, the drilling fluid having a precipitant added therein to form a precipitate with at least a portion of the polymer; and an analyzer to determine an amount of the polymer within the sample, the analyzer being a turbidity meter, a gravimetric weight analysis device, a spectrophotometer, a volumetric analysis device or combinations thereof.

7. The system of paragraph 6, wherein the precipitant is a polyphenolic compound, zinc compound or combinations thereof.

8. The system of paragraphs 6 or 7, further comprising a solids removal device to remove solids from the drilling fluid before the precipitant is added and before the analyzer determines the amount of polymer within the sample.

9. The system of any of paragraphs 6-8, further comprising, in response to the determined concentration of polymer, adding additional polymer to the drilling fluid.

10. The system of any of paragraphs 6-9, wherein the polymer is a linear, crosslinked, or branched polyvinlypyrrolindone (“PVP”) homopolymer or a linear, crosslinked or branched co-polymer containing at least a vinylpyrrolidone monomer.

11. A method to determine a concentration of a polymer in fluid, the method comprising providing a fluid comprising an aqueous base fluid and a polymer; adding a precipitant to the fluid to form a precipitate with at least a portion of the polymer; and determining a concentration of polymer in the fluid by performing at least one of: optically interacting electromagnetic radiation with the precipitate to generate scattered light, wherein the concentration of the polymer is determined based upon the scattered light; gravimetrically measuring the precipitate; spectrally measuring the precipitate; or volumetrically measuring the precipitate.

12. The method of paragraph 11, wherein the polymer is a linear, crosslinked, or branched polyvinlypyrrolindone (“PVP”) or a linear, crosslinked or branched co-polymer containing at least a vinylpyrrolidone monomer.

13. The method of paragraphs 11 or 12, wherein the precipitant is a polyphenolic compound or zinc.

14. The method of any of paragraphs 11-13, wherein the polyphenolic compound is resorcinol, resorcylic acid, or tannic acid.

15. The method of any of paragraphs 11-14, wherein determining the concentration of the polymer based upon the scattered light comprises correlating the amount of scattered light to an amount of the polymer.

16. The method of any of paragraphs 11-15, wherein a calibration curve is used to correlate the amount of scattered light to the amount of the polymer.

17. The method of any of paragraphs 11-16, wherein volumetrically measuring the precipitate comprises separating the precipitate from the aqueous base fluid; and measuring the separated precipitate.

18. The method of any of paragraphs 11-17, further comprising, in response to the determined concentration of polymer, adding additional polymer to the fluid.

19. The method of any of paragraphs 11-18, further comprising removing solids from the fluid before the precipitant is added.

20. The method of any of paragraphs 11-19, wherein the fluid is a drilling fluid. Furthermore, the exemplary methodologies described herein may be implemented by a system including processing circuitry or a non-transitory computer program product including instructions which, when executed by at least one processor, causes the processor to perform any of the methodology described herein.

Although various embodiments and methodologies have been shown and described, the invention is not limited to such embodiments and methodologies and will be understood to include all modifications and variations as would be apparent to one skilled in the art. Therefore, it should be understood that the invention is not intended to be limited to the particular forms disclosed. Rather, the intention is to cover all modifications, equivalents and alternatives falling within the spirit and scope of the invention as defined by the appended claims.

What is claimed is:

1. A method to determine a polymer concentration in an aqueous drilling fluid, the method comprising:
obtaining a sample of a drilling fluid comprising an aqueous base fluid and a polymer comprising a vinylpyrrolidone monomer;
adding a precipitant to the sample to form a precipitate comprising at least a portion of the polymer; and
determining an amount of the polymer within the drilling fluid by performing at least of a turbidity analysis of the sample, a gravimetric weight analysis of the precipitate, a volumetric analysis of the precipitate or combinations thereof.

2. The method of claim 1, wherein the precipitant is selected from polyphenolic compounds, zinc compounds or combinations thereof.

3. The method of claim 1, wherein determining the amount of polymer comprises:
separating the precipitate from the aqueous base fluid by removing solids from the drilling fluid; and
measuring a turbidity of the sample.

4. The method of claim 1, further comprising, in response to the determined concentration of the polymer, adding additional polymer to the drilling fluid.

5. The method of claim 1, wherein the polymer is a linear, crosslinked, or branched polyvinlypyrrolindone (“PVP”) homopolymer or a linear, crosslinked or branched co-polymer containing at least a vinylpyrrolidone monomer.

6. A system to determine a concentration of vinlypyrrolindone in drilling fluid, the system comprising:
a vessel to obtain a sample of drilling fluid comprising an aqueous base fluid and a polymer comprising a vinylpyrrolidone monomer, the drilling fluid having a precipitant added therein to form a precipitate with at least a portion of the polymer; and
an analyzer to determine an amount of the polymer within the sample, the analyzer being a turbidity meter, a gravimetric weight analysis device, a spectrophotometer, a volumetric analysis device or combinations thereof.

7. The system of claim 6, wherein the precipitant is a polyphenolic compound, zinc compound or combinations thereof.

8. The system of claim 6, further comprising a solids removal device to remove solids from the drilling fluid before the precipitant is added and before the analyzer determines the amount of polymer within the sample.

9. The system of claim 6, further comprising, in response to the determined concentration of polymer, adding additional polymer to the drilling fluid.

10. The system of claim 6, wherein the polymer is a linear, crosslinked, or branched polyvinlypyrrolindone (“PVP”) homopolymer or a linear, crosslinked or branched co-polymer containing at least a vinylpyrrolidone monomer.

11. A method to determine a concentration of a polymer in fluid, the method comprising:
providing a fluid comprising an aqueous base fluid and a polymer;
adding a precipitant to the fluid to form a precipitate with at least a portion of the polymer; and
determining a concentration of polymer in the fluid by performing at least one of:
optically interacting electromagnetic radiation with the precipitate to generate scattered light, wherein the concentration of the polymer is determined based upon the scattered light;
gravimetrically measuring the precipitate;
spectrally measuring the precipitate; or
volumetrically measuring the precipitate.

12. The method of claim 11, wherein the polymer is a linear, crosslinked, or branched polyvinlypyrrolindone (“PVP”) or a linear, crosslinked or branched co-polymer containing at least a vinylpyrrolidone monomer.

13. The method of claim 11, wherein the precipitant is a polyphenolic compound or zinc.

14. The method of claim 13, wherein the polyphenolic compound is resorcinol, resorcylic acid, or tannic acid.

15. The method of claim 11, wherein determining the concentration of the polymer based upon the scattered light comprises correlating the amount of scattered light to an amount of the polymer.

16. The method of claim 15, wherein a calibration curve is used to correlate the amount of scattered light to the amount of the polymer.

17. The method of claim 11, wherein volumetrically measuring the precipitate comprises:
separating the precipitate from the aqueous base fluid; and
measuring the separated precipitate.

18. The method of claim 11, further comprising, in response to the determined concentration of polymer, adding additional polymer to the fluid.

19. The method of claim 11, further comprising removing solids from the fluid before the precipitant is added.

20. The method of claim 11, wherein the fluid is a drilling fluid.
